(19) United States
(12) Patent Application Publication
(10) Pub. No.: US 2026/0363679 A1
(43) Pub. Date: Nov. 19, 2026

(54) BIOPSY DEVICES AND METHODS

(71) Applicant: BARD PERIPHERAL VASCULAR, INC., Franklin Lakes, NJ (US)

(72) Inventors: Nicholas C. PETERSON, Mesa, AZ (US); Zachary K. WILLIS, Mesa, AZ (US)

(73) Assignee: BARD PERIPHERAL VASCULAR, INC., Franklin Lakes, NJ (US)

(21) Appl. No.: 19/136,805

(22) PCT Filed: Jan. 9, 2023
(86) PCT No.: PCT/US2023/060321

§ 371 Date: Jun. 6, 2025

(51) Int. Cl. A61B 10/02

(52) U.S. Cl. CPC A61B 10/0275; A61B 10/0283; A61B 2010/0208

(57) ABSTRACT

A biopsy device comprises a handle with one or more user input devices and a biopsy needle assembly connected to the handle with a retractable cannula extending distally from the handle and retractable into the handle; a stylet longitudinally positioned within the retractable cannula, a sample passage defined between an inner surface of the retractable cannula and an outer surface of the stylet, the stylet defining a sample recess; and a conductive needle tip coupled to a distal end of the stylet within the mounting portion, wherein operation of the conductive needle tip heats a remaining tissue in contact therewith, and the stylet substantially insulates proximal heat conduction from the conductive needle tip.

### BACKGROUND

### Field

The present disclosure relates generally to biopsy devices and methods, and more specifically, to biopsy devices and methods having cauterizing capabilities.

### Technical Background

Biopsy devices are used to acquire tissue samples for a variety of medical issues. For example, when imaging exams cannot clearly define an abnormality such as a nodule, a biopsy may be necessary. Typical biopsy procedures may lead to unwanted bleeding and/or extended healing times.

Accordingly, new biopsy devices and methods are desirable that reduce unwanted bleeding and/or reduce healing time.

### SUMMARY

In one embodiment, a biopsy device includes a handle and a biopsy needle assembly connected to the handle. The handle includes one or more user input device. The biopsy needle assembly includes a retractable cannula, a stylet, and a conductive needle tip. The retractable cannula extends distally from the handle and is retractable into the handle. The stylet is longitudinally positioned within the retractable cannula. A sample passage is defined between an inner surface of the retractable cannula and an outer surface of the stylet. The stylet defines a sample recess. The conductive needle tip is coupled to a distal end of the stylet within the mounting portion. Operation of the conductive needle tip heats a remaining tissue in contact therewith and the stylet substantially insulates proximal heat conduction from the conductive needle tip.

In another embodiment a biopsy device includes a handle including one or more user input devices, a biopsy needle assembly connect to the handle, and a vacuum. The biopsy needle assembly includes a retractable cannula extending distally from the handle and retractable into the handle, a stylet, and a conductive needle tip. The stylet is longitudinally positioned within the retractable cannula, a sample passage is defined between an inner surface of the retractable cannula and an outer surface of the stylet, and the stylet defining a sample recess. The conductive needle tip is coupled to a distal end of the stylet within the mounting portion, wherein operation of the conductive needle tip heats a remaining tissue in contact therewith, and the stylet substantially insulates proximal heat conduction from the conductive needle tip. The vacuum is fluidically coupled to the retractable cannula.

In yet another embodiment, a method for assembling a biopsy device includes coupling a conductive needle tip within a mounting portion of a stylet and longitudinally positioning the stylet within a retractable cannula. A sample passage is defined between an inner surface of the retractable cannula and an outer surface of the stylet. The method further includes retractably attaching the retractable cannula to a handle. The conductive needle tip may be selectively heated, and the stylet substantially insulates proximal heat conduction from the conductive needle tip.

In yet another embodiment, a method for performing a biopsy procedure includes advancing a biopsy device to a targeted biopsy site. The biopsy device includes a retractable cannula extending distally from a handle and retractable into the handle, a stylet longitudinally positioned within the retractable cannula, a sample passage defined between an inner surface of the retractable cannula and an outer surface of the stylet, the stylet defining a sample recess, and a conductive needle tip coupled to a distal end of the stylet within the mounting portion, wherein operation of the conductive needle tip heats a remaining tissue in contact therewith, and the stylet substantially insulates proximal heat conduction from the conductive needle tip. The method further includes retracting the retractable cannula, advancing the retractable cannula to obtain a biopsy sample, and actuating the conductive needle tip to cauterize the targeted biopsy site.

It is to be understood that both the foregoing general description and the following detailed description describe various embodiments and are intended to provide an overview or framework for understanding the nature and character of the claimed subject matter. The accompanying drawings are included to provide a further understanding of the various embodiments, and are incorporated into and constitute a part of this specification. The drawings illustrate the various embodiments described herein, and together with the description serve to explain the principles and operations of the claimed subject matter.

### BRIEF DESCRIPTION OF THE DRAWINGS

The above-mentioned and other features and advantages of this disclosure, and the manner of attaining them, will become more apparent and will be better understood by reference to the following description taken in conjunction with the accompanying drawings, wherein:

FIG. 1 schematically depicts a perspective view of a biopsy device in a cannula extended state, according to one or more embodiments shown and described herein;

FIG. 2 schematically depicts a perspective view of the biopsy device of FIG. 1 in a cannula retracted state, according to one or more embodiments shown and described herein;

FIG. 3 schematically depicts a distal end of the biopsy device of FIG. 1 in the cannula retracted state, according to one or more embodiments shown and described herein;

FIG. 4 schematically depicts a cross-sectional view of the distal end of a biopsy device of FIG. 1, according to one or more embodiments shown and described herein;

FIG. 5 schematically depicts a perspective view of a biopsy device tip having an externally threaded connection, according to one or more embodiments shown and described herein;

FIG. 6 schematically depicts a perspective view of a biopsy device stylet having an internally threaded connection, according to one or more embodiments shown and described herein;

FIG. 7 schematically depicts a perspective view of a distal end of a biopsy device similar to FIG. 4 but including a spring pin connection and heat shield, according to one or more embodiments shown and described herein;

FIG. 8 schematically depicts a transparent perspective view of a biopsy device showing a vacuum lumen pathway and heating core lead wires, according to one or more embodiments shown and described herein;

FIG. 9 depicts a flowchart of an illustrative method for using a biopsy device to collect a tissue sample and cauterize remaining tissue, according to one or more embodiments shown and described herein;

FIG. 10A depicts the biopsy device of FIG. 1 in an initial position prior to tissue sample collection, according to one or more embodiments shown and described herein;

FIG. 10B depicts the biopsy device of FIG. 10A inserted at a target site, according to one or more embodiments shown and described herein;

FIG. 10C depicts the outer cannula of the biopsy device of FIG. 10B in a retracted position at the target site with the outer cannula retracted, according to one or more embodiments shown and described herein;

FIG. 10D depicts the outer cannula of FIG. 10C in an extended position at the target site, according to one or more embodiments shown and described herein;

FIG. 10E depicts the biopsy device of FIG. 10D being removed from the target site, according to one or more embodiments shown and described herein; and

FIG. 11 depicts a flowchart of an illustrative method for assembling a biopsy device, according to one or more embodiments shown and described herein.

Corresponding reference characters indicate corresponding parts throughout the several views. The exemplifications set out herein illustrate embodiments of the disclosure, and such exemplifications are not to be construed as limiting the scope of the disclosure in any manner.

### DETAILED DESCRIPTION

The present disclosure overcomes deficiencies in biopsy procedures as described above by including a cauterizing tip that lessens bleeding and/or improves healing rates following a biopsy procedure without need of separate devices or separate procedures. The present disclosure provides various embodiments of biopsy devices, methods for assembling a biopsy device, and methods for using a biopsy device. In embodiments, the biopsy device includes a metal tip configured to conduct heat and an insulative stylet to prevent backwards or proximal heat travel. The cauterizing heating element is used to close a wound caused by the biopsy needle assembly. The cauterizing heating element generally heats the remaining tissue to a predetermined temperature for a period of time sufficient to cauterize the remaining tissue, thereby reducing bleeding and/or speeding recovery. In addition to aiding in bleeding/recovery, cauterizing can also reduce the impact of seeding or spreading of cancerous tissue during device removal. These and additional embodiments and benefits will be described in greater detail below.

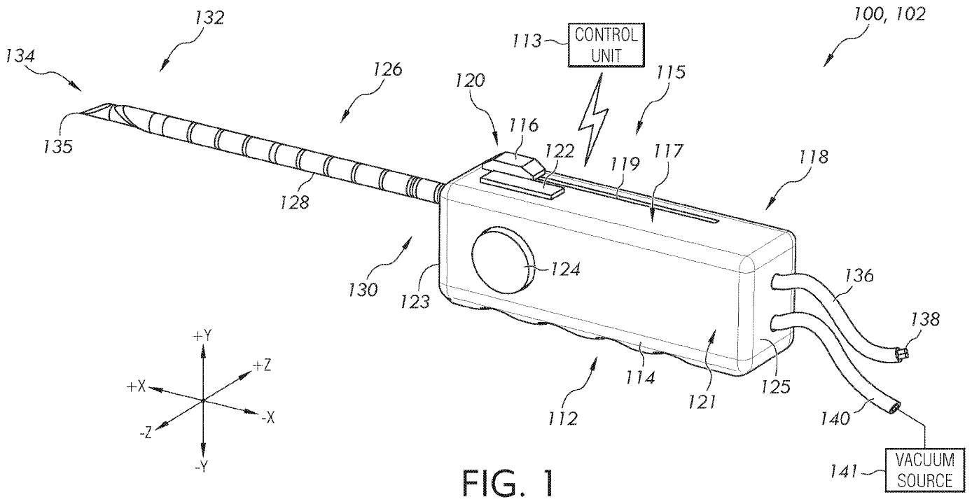

FIGS. 1-3 generally depict a biopsy device 100 including a handle 112 and a needle assembly 126 connected to the handle 112. For example, a proximal end 130 of the needle assembly 126 may be coupled to a distal end 123 of the handle 112. As will be described in greater detail below, the needle assembly 126 generally includes a retractable cannula 128, a stylet 142, and a conductive needle tip 134. The needle assembly 126 can be any gauge (e.g., 14-gauge, 16-gauge, 18-gauge, 20-gauge) suitable for biopsying a tissue sample. It is noted that while embodiments are shown with a number of components, a greater or fewer number of components may be included without departing from the scope of the present disclosure.

The handle 112 may generally include handle body 114 and one or more user input devices 115 or controls mounted to the handle body 114. The handle body 114 may generally enclose any number of electronics, wires, tubing, or the like. The one or more user input devices 115 can be positioned on any surface of the handle body 114. The one or more user input devices 115 may include a number of toggles, switches, slides, buttons, touchscreens, etc. In the depicted embodiment, the one or more user input devices 115 includes slider 116. Though the slider 116 is illustrated as located along a top surface 117 of the handle body 114, the slider 116 may be positioned along any surface. The slider 116 may be configured to slide along a slot 119 in the +/−X direction of the depicted coordinate axes from a proximal end 118 of the slot 119 towards a distal end 120 of the slot 119. As will be described in greater detail herein, the slider 116 may be operatively coupled to the retractable cannula 128 and used to retract or extend the retractable cannula 128 relative to the handle 112. In some embodiments, the slider 116 merely indicates to a user a position of the retractable cannula 128 relative to the handle 112. For example, and as described further below the slider 116 and/or the retractable cannula 128 may be coupled to a motor or the like that moves the retractable cannula 128 and/or the slider 116.

In embodiments, the one or more user input devices 115 may further include a firing button 122, a retract button (not depicted), and/or a heat button 124. In embodiments, as opposed to manually sliding the retractable cannula 128, the retractable cannula 128 may be coupled to any number of motors, gears, or the like configured to engage the retractable cannula 128 to move the retractable cannula 128 between retracted and extended positions. For example, retract button may be operated by a user to cause the retractable cannula 128 to be retracted by a motor or other type actuator. The firing button 122 may then be used to fire the retractable cannula 128 forward, to for example, biopsy tissue. For example, the retractable cannula 128 may be spring loaded to fire forward upon actuation of the firing button 122. It is noted that while the firing button is illustrated as being positioned on the top surface 117 of the handle 112, the firing button 122 may be positioned on any surface of the handle body 114. It is further noted that while the firing button 122 is indicated as being a button, it may be any type of user input devices 115 including, but not limited to a toggle, switch, touch screen, etc. Additional buttons or controls may be placed on handle body 114.

In some embodiments, the handle 112 houses a control unit 113 communicatively coupled to the one or more user input devices 115, and a signal from the one or more user input devices 115 causes the control unit 113 to operate various functions of the biopsy device 100 such as tissue cutting, vacuum, and/or heating. The control unit 113 can include any number of processors, chips, memories, storing non-transitory computer readable media, or the like.

The processor includes, for example, a microprocessor, a non-transitory electronic memory, and associated circuitry, such as an input/output interface, clock, buffers, etc. The memory is non-transitory electronic memory, and may include volatile memory, such as random access memory (RAM), and/or non-volatile memory, such as read only memory (ROM), electronically erasable programmable ROM (EEPROM), NOR flash memory, NAND flash memory, etc. The memory can be used to store operational information associated with retractable cannula 128 in order to retract the retractable cannula 128, advance the retractable cannula 128 to obtain a biopsy sample, and/or actuate the conductive needle tip 134 to cauterize the targeted biopsy site by way of heat button 124, based on user input received via the one or more user input devices, and/or logic stored on the memory.

As will be described in greater detail herein, the heat button 124 may be used to operate or provide heat to the conductive needle tip 134. For example, the heat button 124 may operate to close a circuit between, for example, a power source (e.g., a DC or AC power source) and the conductive needle tip 134, whereby heat is generated at the conductive needle tip 134. It is noted that while the heat button 124 is illustrated on a side surface 121 of the handle body 114, the heat button 124 may be positioned along any surface of the handle body 114. It is further noted that while the heat button 124 is indicated as being a button, it may be any type of user input devices 115 including, but not limited to a toggle, switch, touch screen, etc. Having the user input devices on the handle 112 may optionally improve usability of the biopsy device 100, as a user may hold the handle to position the biopsy device 100 at a desired location and then may make use of the functions of biopsy device 100 without moving their hand away from the handle 112.

In some embodiments, an energy delivery device is positioned within the handle 112 and operatively coupled to the conductive needle tip 134. For example, the energy delivery device may be a battery positioned within or attached to the handle 112. Having an energy delivery device positioned within the handle 112 may optionally reduce a number of wires extending from the biopsy device 100, which may improve the ease of handling of the biopsy device 100. Alternatively, the handle may connect to a power source (e.g., an outlet or generator) to provide power for the various functions of the biopsy device 100. Having a connection to a power source may optionally make biopsy device 100 lighter (by eliminating the need for a battery) and may also avoid the chance of encountering a drained battery. In embodiments, the handle 112 may be coupled to power sources and/or vacuum sources (e.g., vacuum source 141) via a heating line 136 and/or vacuum tube 140. For example, the heating line 136 and/or the vacuum tube 140 may couple to a proximal end 125 of the handle 112. The heating line 136 may contain one or more heating core lead wires 138, which, as described in greater detail below, may extend to the conductive needle tip 134 to allow the conductive needle tip 134 to be heated electrically by coupling the conductive needle tip 134 to a power source.

In embodiments, one or more lumens of the needle assembly 126 may be fluidically coupled to vacuum source 141 (e.g. a vacuum pump) via the vacuum tube 140 to aid in sample acquisition and/or retrieval. In some embodiments, the vacuum tube 140 can be fluidly coupled to a sample collection basket (not shown) near the proximal end of biopsy device 100. The sample collection basket can be a fluid container of any shape. The sample collection basket can also include a filter to separate tissue sample 214 and fluid. In some embodiments vacuum source 141 may be integrated in the handle 112 making the biopsy device 100 easier to handle and position.

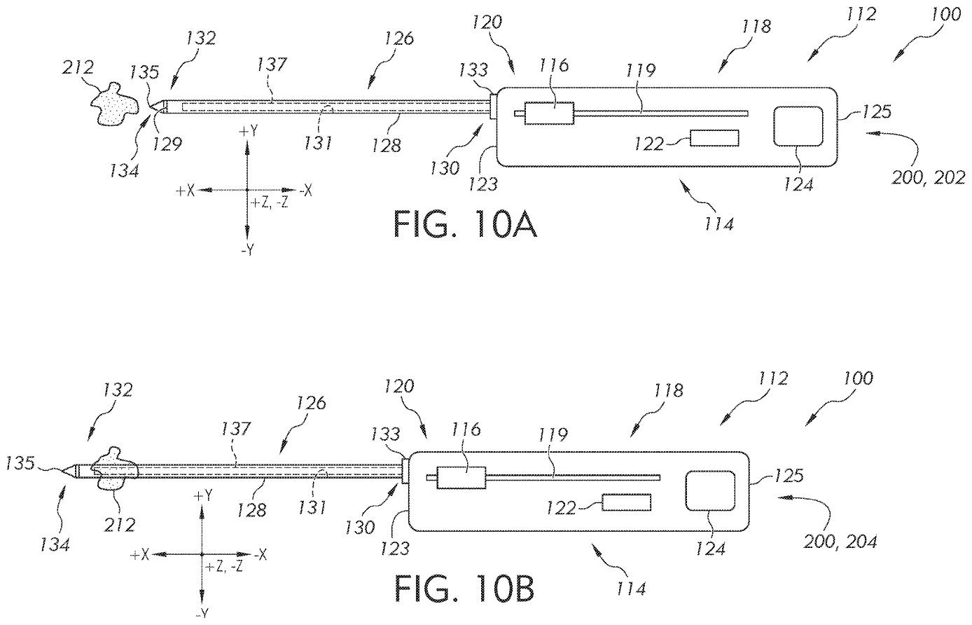

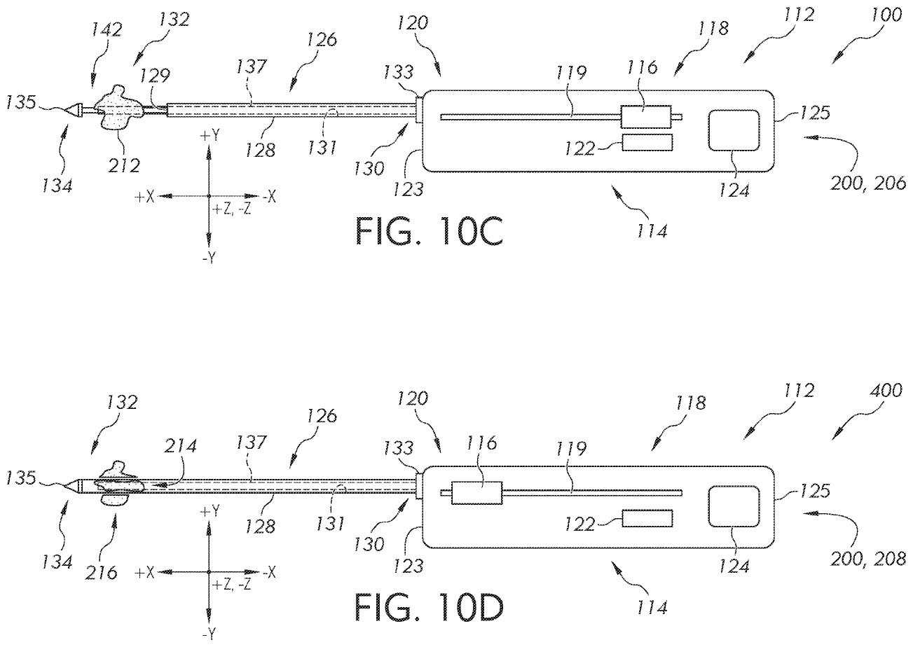

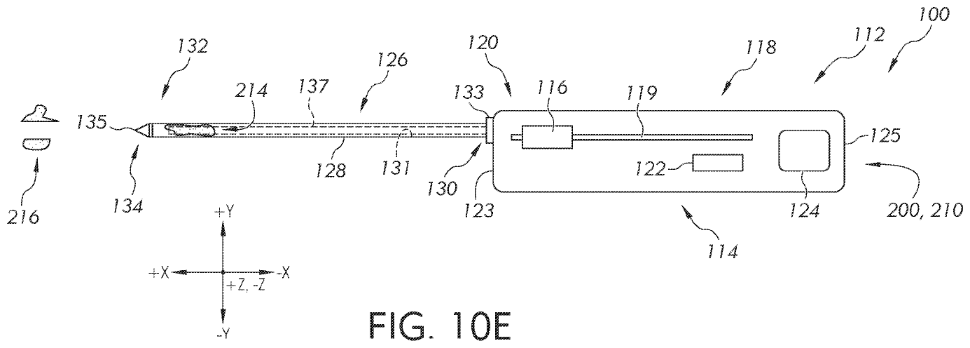

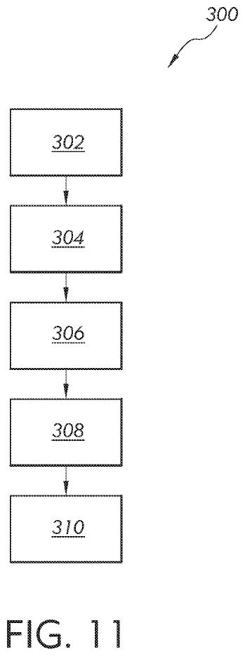

As noted above, the handle 112 is coupled to the needle assembly 126. For example, as shown in FIGS. 10A-E, the needle assembly 126 may be mounted to the handle 112 via an aperture 133 formed within the handle body 114. For example, the needle assembly 126 may extend distally through the aperture 133 from handle 112 to a distal end 132. As noted above, the needle assembly 126 generally includes the retractable cannula 128, the stylet 142, and the conductive needle tip 134.

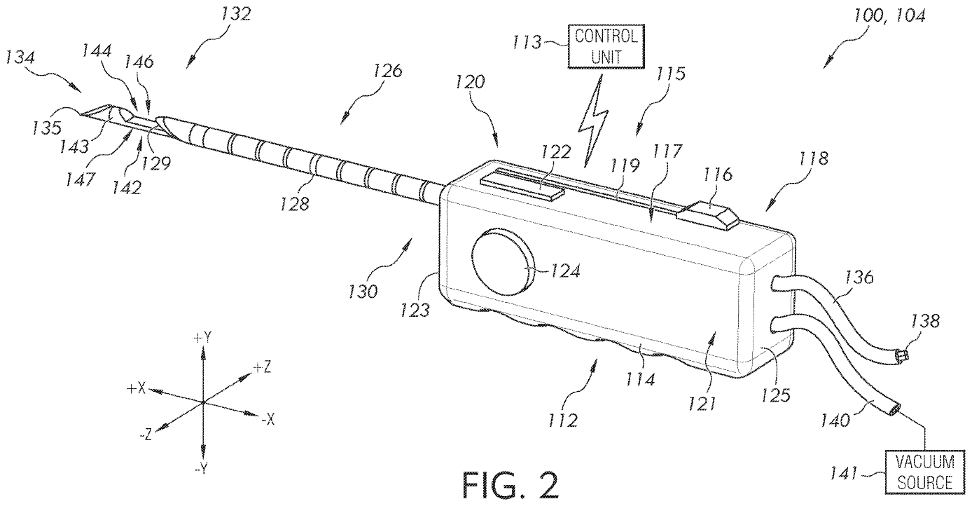

The retractable cannula 128 extends distally from the handle 112 and is retractable into the handle 112. That is, the retractable cannula 128 is retractable and/or extendable relative to the handle 112. Accordingly, the retractable cannula 128, may be coupled to any number of motors, gears, sliders, or the like, configured to retract or extend the retractable cannula 128 relative to the handle 112. FIG. 1 shows biopsy device 100 in a first position 102 where the retractable cannula 128 is fully extended toward the distal end 132. FIG. 2 illustrates the retractable cannula 128 in a second position 104 retracted proximally along the +/−X axis of the depicted coordinate axes from the distal end 132 into the handle body 114. As noted above, the slider 116 and/or the firing button 122 may be operable to cause the retractable cannula 128 to retract and/or extend as desired by a user.

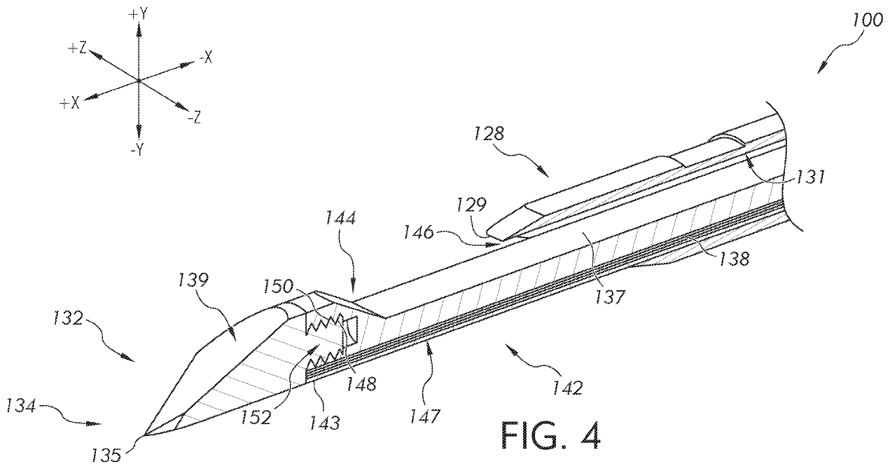

The retractable cannula 128 may be an elongate tube defining cannula lumen 137 extending therethrough, such as best depicted in FIG. 4. The retractable cannula 128 may define a sharp cutting edge 129 at its distal end. As will be described in greater detail herein, the retractable cannula 128 may sever tissue 212 with the sharp cutting edge 129. In some embodiments, the retractable cannula 128 may be formed of any suitable material for performing a biopsy procedure, such as stainless steel, titanium, a nickel alloy, a composite material, a ceramic material, or the like.

The stylet 142 is longitudinally positioned within the retractable cannula 128. That is the stylet 142 is positioned within the cannula lumen 137 of the retractable cannula 128. The stylet 142 may be an elongate member having an outer surface 147. A sample passage 146 is defined between an inner surface 131 of the retractable cannula 128 and an outer surface 147 of the stylet 142. The stylet 142 may define a sample recess 144 proximate to a distal end 143 of the stylet 142, wherein the sample passage 146 is defined between the sample recess 144 and the inner surface 131 of the retractable cannula 128. As will be described in greater detail below, the retractable cannula 128 may be retracted a desired amount to expose the sample recess 144 for sample collection.

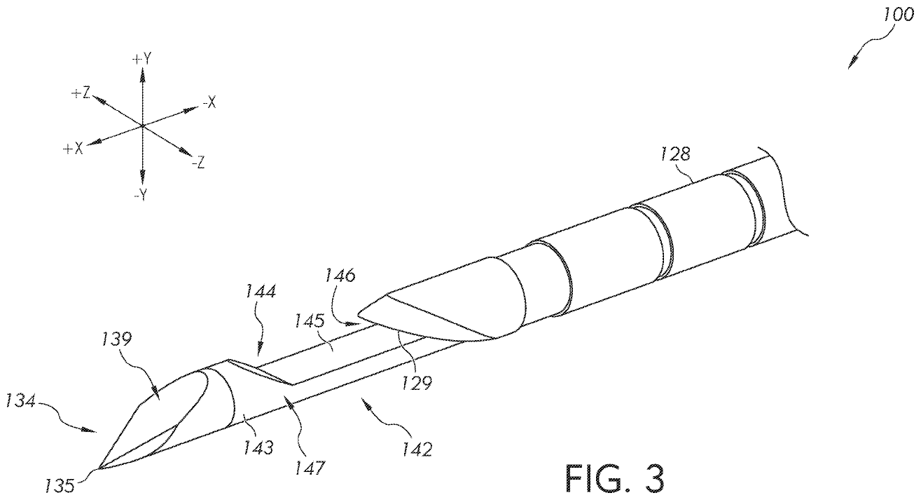

FIG. 3 shows an enlarged distal end 132 of the biopsy device 100, particularly illustrating the stylet 142. In embodiments, the stylet 142 may be formed of any number of materials. For example, the stylet 142 may be an injection-molded composite, metal, or the like. In embodiments wherein the stylet 142 is formed of a composite material, ceramic or metal reinforcing rib(s) may be added to the stylet to provide additional rigidity. Additional rigidity may make it easier to advance the stylet 142.

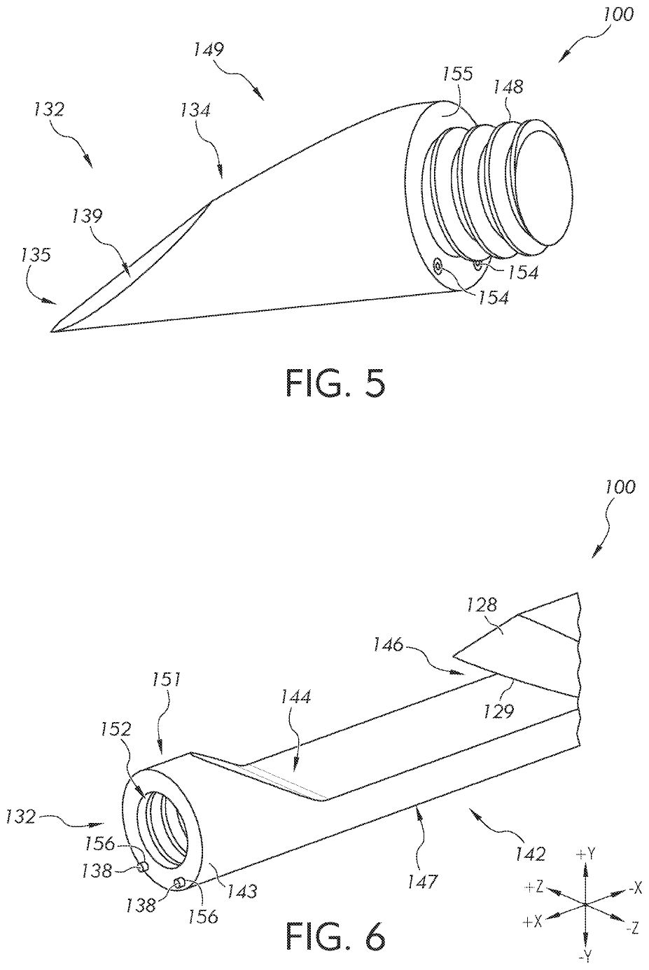

Referring particularly to FIG. 6, the stylet 142 may define a mounting portion 151 formed at the distal end 143 of the stylet 142. The mounting portion 151 may be sized to receive and mount the conductive needle tip 134 to the stylet 142. For example, the mounting portion 151 may have an internally threaded connection 150 to threadingly engage an externally threaded connection 148 of the conductive needle tip 134 shown in FIG. 5. In some embodiments, the conductive needle tip 134 is glued or crimped into the mounting portion 151. In other embodiments, the conductive needle tip 134 may be received within the mounting portion 151 and held in place via an inference fit or friction fit between the conductive needle tip 134 and the stylet 142. In some embodiments, the stylet 142 may instead define a stub (threaded or not) received within the conductive needle tip and coupled thereto via crimping, adhesive, threaded engagement, of the like. Having the conductive needle tip 134 as a separate piece, mounted to the mounting portion 151 of the stylet 142 may optionally allow the conductive needle tip 134 to be replaced between uses of the biopsy device 100. That is, a used, blunted, conductive needle tip 134 may be replaced with a fresh, sharp, conductive needle tip 134. This may optionally reduce waste and increase the re-usability of the biopsy device 100.

Still referring to FIG. 6, the stylet 142 may define one or more wire lumens 156 extending longitudinally within the stylet 142. The one or more wire lumens 156 may be sized to receive the one or more heating core lead wires 138 for heating the conductive needle tip 134. The one or more wire lumens 156 may include two or more lumens each receiving heating core lead wires 138. The one or more wire lumens 156 may extend a length of the stylet 142 through the distal end 143. The one or more heating core lead wires 138 may be electrically coupled to an energy delivery device (e.g., a battery, or other electrical source). As noted above, in some embodiments the energy delivery device may be positioned within the handle 112. In any case, the one or more heating core lead wires 138 provide a conductive pathway through the stylet 142 to the conductive needle tip 134. In particular, the heating core lead wires 138 are coupled to the conductive needle tip 134 which heats as current travels through stylet 142 via the heating core lead wires 138, to the conductive needle tip 134. In some embodiments, the conductive needle tip may have a resistive heating element provided therein (or be a resistive heating element). Accordingly, current passing through the resistive heating element causes heat to be generated, which is conducted through the conductive needle tip 134. Accordingly, the conductive needle tip 134 may be heated to a cauterizing temperature (150 °C or greater). Providing the wire lumens 156 may optionally help to prevent damage to the heating core lead wires 138 and/or allow easier passage of a biopsy sample, by keeping the heating core lead wires 138 separate from the sample passage 146. Though the heating core lead wires 138 are depicted as extending through the stylet 142, alternatively, heating core lead wires 138 may extend through sample passage 146 of retractable cannula 128. It is noted that heating core lead wires 138 can also be held within electrically insulated sleeves. In another alternative, lead core wires 138 may be wrapped circumferentially around the stylet 142, and attached to an RF generator to rapidly reach cauterizing temperatures.

The stylet 142 in some embodiments may be a composite stylet formed of a composite material, metals, and/or ceramics. For example, composite materials for stylet 142 could include PEEK, PPS, Ultem Polyetherimide (PEI), Noryl PPO, Polysulfone, PTFE, Vespel, Torlon (polyamide-imide PAI) among others. The stylet 142 in some embodiments may be a composite stylet formed of a metal such as stainless steel, titanium, a nickel alloy, or the like. Where the stylet is formed of metal, it is noted the heating core lead wires 138 may be insulated thermally and/or electrically to prevent heating of the stylet 142. The stylet 142, such as where the stylet 142 is formed of a composite or ceramic, may prevent proximal heat conduction from the conductive needle tip 134. This may optionally prevent unwanted heat transfer from the conductive needle tip 134, through the stylet 142, and to a biopsy sample. Where the stylet 142 is formed of ceramic, to ease manufacturing an exposed wire groove may be formed along a bottom surface of stylet 142 for receiving the heating core lead wires 138. In embodiments, such wire groove may be filled with an epoxy or UV cured resin after wires are secured in placed. In various embodiments, the stylet 142 may be formed via molding (e.g., injection molding) or other machining techniques. In embodiments where the stylet 142 is formed of an electrically conductive material (e.g., metal) the stylet 142 may not have wire lumens and may instead act as a conductor between an energy delivery device (such as described above) and the conductive needle tip 134. In such embodiments, one or more insulating material may be coated or applied to the stylet 142 to prevent unwanted heat transfer from the stylet 142 to a captured tissue sample 214.

As noted above, the needle assembly 126 further includes the conductive needle tip 134 coupled to a distal end of the stylet 142 within the mounting portion 151. As will be described in greater detail below, operation of the conductive needle tip 134 heats remaining tissue 216 in contact with the conductive needle tip 134, and the stylet 142 may substantially insulate proximal heat conduction from the conductive needle tip 134, thereby insulating the biopsied tissue sample 214 from the conductive needle tip 134.

The conductive needle tip 134 may be formed of a metallic material (e.g., silver, copper, aluminum, beryllium copper, etc.) which may be heated via passing current therethrough (such as provided via the stylet 142 or one or more heating core lead wires 138 described above). The conductive needle tip 134 may include a mounting portion 151 and a piercing portion 149 coupled to the mounting portion 151. For example the mounting portion 151 and the piercing portion 149 may be integral with one another or coupled to one another via any suitable manufacturing technique (e.g., welding, brazing, etc.).

As shown in FIG. 5 and. 6, the mounting portion 151 may be configured to receive conductive needle tip 134. For example, the mounting portion 151 may have an internally threaded connection 150 to threadingly engage an externally threaded connection 148 of conductive needle tip 134 within the mounting portion 151. (As noted above, conductive needle tip may instead receive a stub of the stylet 142 and coupled thereto.) In some embodiments, the conductive needle tip 134 is releasably and electrically coupled to the stylet 142 via a spring-loaded pin. For example, utilizing a spring-loaded pin, may eliminate a need for perfect alignment between the two components during assembly.

The piercing portion 149 may have a sharp distal tip 135 for piercing tissue 212. In embodiments, the piercing portion 149 includes a proximal face 155 defining one or more inlets for receiving the one or more heating core lead wires 138 passing through the stylet. In embodiments, the one or more inlets cause the one or more heating core lead wires 138 to couple to a heating element (e.g., a resistive element) embedded within the conductive needle tip 134. In some embodiments, the conductive needle tip 134 itself is configured to receive current and as current passes through the conductive needle tip 134, the conductive needle tip 134 heats. The conductive needle tip 134 may be configured to heat to a temperature sufficient to cauterize remaining tissue 216 (e.g., such as about 150 °C or greater). The distal part of conductive needle tip 134 can be tapered to more easily penetrate a tissue 212.

In some embodiments, the conductive needle tip 134 can be treated by sandblasting or the like. Sandblasting can provide echogenic features for tracking progress under ultrasound. This may optionally allow easier tracking of the conductive needle tip 134 as the stylet 142 and retractable cannula 128 is advanced.

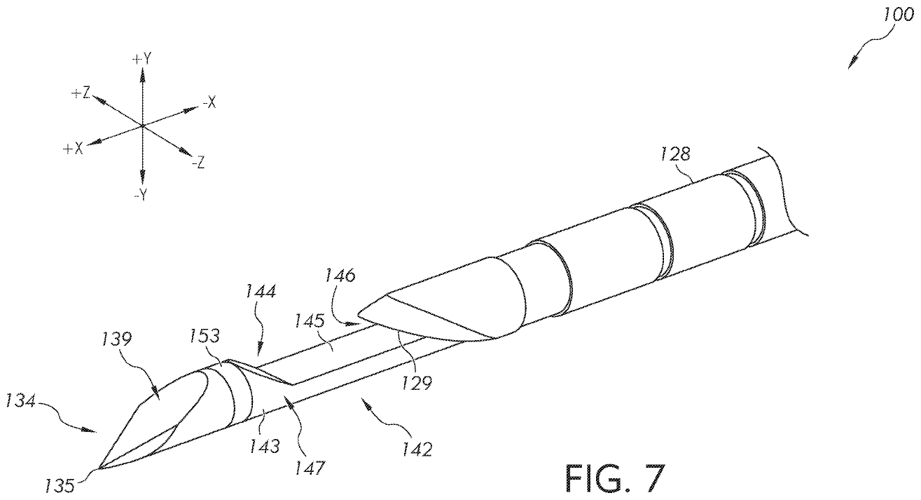

In some embodiments and with reference to FIG. 7, a heat shield 153 may be positioned between the conductive needle tip 134 and the stylet 142, such as distal to the sample recess 144 to shield tissue sample from heat generated by the conductive needle tip 134. Accordingly, proximal heat transfer through the biopsy device 100 may be avoided. In some embodiments, the heat shield 153 may be a thin spacer between conductive needle tip 134 and the stylet 142 made from a bio-compatible or bio-safe polymer or ceramic. The heat shield 153 may be formed of any suitable material, such as an alumina or zirconia insulative ceramics, or a thermally insulative composite like PEEK, PTFE, ABS, Polyimide, PVC, etc. The heat shield 153 may optionally assist in avoiding unwanted heating of a collected sample.

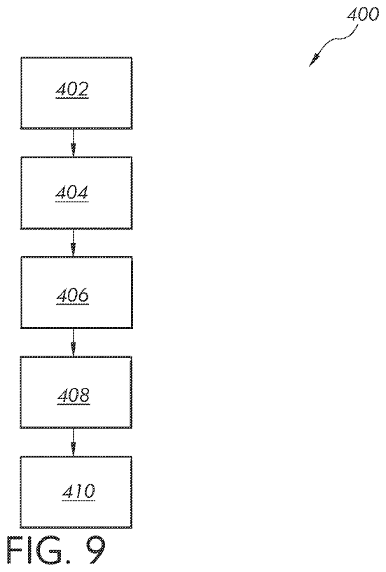

Referring now to FIG. 9, a method 400 for using a biopsy device 100 to collect a tissue sample 214 and cauterize remaining tissue 216 is generally depicted. The method may include a greater or fewer number of steps in varying orders without departing from the scope of the present disclosure. At block 402 the method 400 includes advancing the biopsy device 100 to a targeted biopsy site. For example, as depicted in FIGS. 10A and 10B, the biopsy device 100 is illustrated as being advanced through a target tissue 212.

Referring again to FIG. 9, at block 404, the method 400 includes retracting the retractable cannula 128 proximally (e.g., in the −X direction of the depicted coordinate axes as depicted in FIG. 10C), such as by using user input devices 115 on the handheld device, as described above. By retracting the retractable cannula 128, the sample recess 144 is revealed, which allows target tissue to migrate into the sample recess 144.

Still referring to FIG. 9, at block 406 the method 400 further includes advancing the retractable cannula 128 distally as depicted in FIG. 10D in the +X direction of the depicted coordinate axes to sever a biopsy sample with the retractable cannula 128.

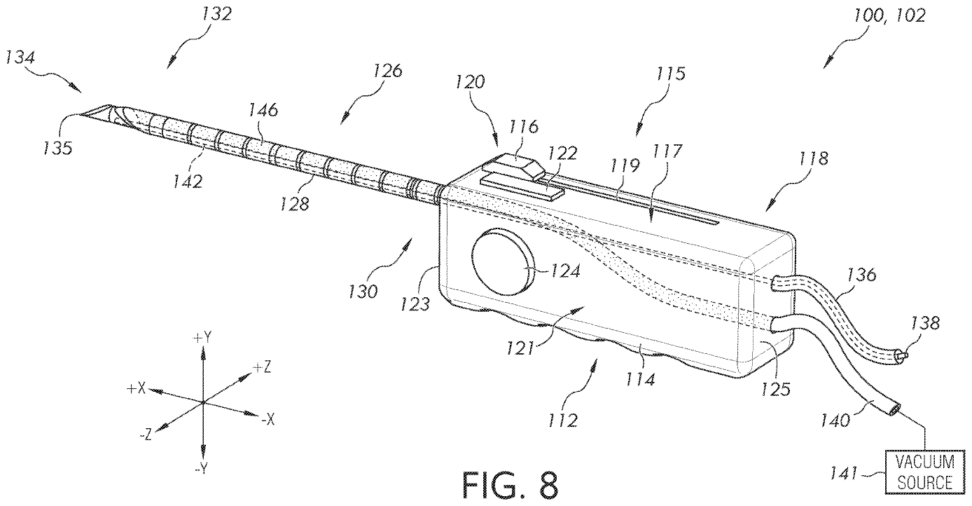

Still referring to FIG. 9, at block 408 the method 400 further includes operating the vacuum source 141 to advance the biopsy sample along a sample passage 146 to a sample collection basket. Accordingly, in some embodiments, method 400, may include further steps such as, but not limited to, coupling the biopsy device 100 to a vacuum source 141. As shown in FIG. 8, the vacuum source 141 advances the biopsy sample from sample passage 146 through handle body 114 via vacuum tube 140. Advancing the tissue sample 214 away from the conductive needle tip 134 may optionally help avoid undesirable heating of the biopsy sample during the cauterization procedure. Use of the vacuum source 141 may optionally allow collection/study of the tissue sample 214 while the distal end of the biopsy device is still located proximate the target tissue 212. This may optionally allow a medical professional to determine whether a sufficiently large tissue sample 214 has been collected, or if more needs to be collected from that same tissue 212.

Referring to FIG. 9 at block 410, the method 400 may further include activating the conductive needle tip to cauterize the resulting biopsy site, as described above. For example, the heat button 124 may be actuated by a user after the biopsy sample is captured to cause the conductive needle tip 134 to heat up and cauterize the resulting wound. In some embodiments, the conductive needle tip 134 may be retracted slightly (i.e. in the proximal −X direction) before cauterization, so that the conductive needle tip 134 cauterizes precisely at the region where the tissue was cut. Alternatively, the conductive needle tip 134 heated while it is at its location in FIG. 10D and then slowly retracted (while still heated) to continue to cauterize as it is removed to at least to where the cutting edge 129 is shown in FIG. 10C. This would ensure the remaining tissue 216 is cauterized. After cauterization and/or movement of the biopsy sample via vacuum, the biopsy device 100 may be withdrawn from the biopsy site such as illustrated in FIG. 10E.

Referring now to FIG. 11 a method 300 for assembling a biopsy device is generally depicted. A greater or fewer number of steps, taken in any order, may be included without departing from the scope of the present disclosure. The method includes, at block 302, attaching the conductive needle tip 134 within mounting portion 151 of stylet 142. For example, and as noted above, the conductive needle tip 134 can be threaded at an externally threaded connection 148 to mate with an internally threaded connection 150 of stylet 142 or crimped into a locking insert of stylet 142. In other embodiments, the conductive needle tip 134 may be held in place via a pin or similar fastener. In other embodiments, the conductive needle tip 134 may be held in place via welding or soldering. At block 304, the method includes longitudinally positioning the stylet 142 within a retractable cannula 128. As described above, in embodiments, longitudinally positioning the stylet 142 within the retractable cannula 128 defines a sample passage 146 between an inner surface of the retractable cannula 128 and an outer surface of the stylet 142. At block 306, the method includes retractable attaching the retractable cannula 128 to the handle 112. For example, the handle 112 may be coupled to the retractable cannula 128 of needle assembly 126 at the proximal end of needle assembly 126. For example, the retractable cannula 128 and/or stylet 142 may be received within the aperture 133. In embodiments, the retractable cannula 128 may be operatively coupled within the handle 112 to any number of motors, gearing, actuators or the like to retract and extend the retractable cannula 128 for perform a biopsy operation as described above.

At block 308, the method 300 may include positioning the one or more heating core lead wires 138 within the one or more stylet wire lumens 156. The method 300 may further include coupling the one or more heating core lead wires 138 to a power source such as described herein such that current may be conducted via the one or more heating core lead wires 138 to the conductive needle tip 134.

At block 310, the method 300 may further include coupling the handle 112 to vacuum source 141 that provides suction through the retractable cannula 128. For example, a vacuum tube 140 coupled to handle body 114. Cannula lumen 137 of the retractable cannula 128 is fluidly coupled to the vacuum tube 140. The vacuum tube 140 can be fluidly coupled to a basket (not shown) near the proximal end of biopsy device 100 and the basket can be a fluid container of any shape.

Thus it is seen that devices, systems and methods for assembling and using a biopsy device are provided including the use of a metal tip to conduct heat and an insulative stylet to prevent backwards heat travel. The composite cauterizing-tip cauterizes remaining tissue as the biopsy instrument is extracted, causing less bleeding directly into the biopsy cavity. As noted above, cauterization further reduces the impact of “seeding” of cancerous tissue during device removal.

Embodiments of the present disclosure may be further described with respect to the following numbered aspects:
Aspect 1. A biopsy device comprises a handle comprising one or more user input devices and a biopsy needle assembly connected to the handle. The biopsy needle assembly comprises a retractable cannula extending distally from the handle and retractable into the handle, a stylet longitudinally positioned within the retractable cannula, a sample passage defined between an inner surface of the retractable cannula and an outer surface of the stylet, the stylet defining a sample recess, and a conductive needle tip coupled to a distal end of the stylet within the mounting portion, wherein operation of the conductive needle tip heats remaining tissue in contact therewith, and the stylet substantially insulates proximal heat conduction from the conductive needle tip.
Aspect 2. The biopsy device according to Aspect 1, wherein the handle houses a control unit communicatively coupled to the one or more user input devices, and a signal from the one or more user input devices causes the control unit to operate the conductive needle tip.
Aspect 3. The biopsy device according to any of Aspect 1 to Aspect 2, wherein the retractable cannula comprises a composite material, a ceramic material, or any combination thereof.
Aspect 4. The biopsy device according to any of Aspect 1 to Aspect 3, wherein the stylet comprises a reinforcing rib.
Aspect 5. The biopsy device according to any of Aspect 1 to Aspect 4, further comprises one or more lead wires, wherein the stylet defines one or more wire lumens. The one or more lead wires are positioned within the one or more wire lumens to provide a conductive pathway through the stylet to the conductive needle tip.
Aspect 6. The biopsy device according to any of Aspect 1 to Aspect 5, wherein the conductive needle tip is threaded or crimped into the mounting portion of the stylet.
Aspect 7. The biopsy device according to any of Aspect 1 to Aspect 6, further comprises a heat shield positioned longitudinally between the conductive tip and stylet.
Aspect 8. A biopsy device comprising a handle comprises one or more user input devices and a biopsy needle assembly connected to the handle. The biopsy needle assembly comprises a retractable cannula extending distally from the handle and retractable into the handle, a stylet longitudinally positioned within the retractable cannula, and a sample passage defined between an inner surface of the retractable cannula and an outer surface of the stylet, the stylet defining a sample recess. The biopsy needle assembly further comprises a conductive needle tip coupled to a distal end of the stylet within the mounting portion, wherein operation of the conductive needle tip heats remaining tissue in contact therewith, and the stylet substantially insulates proximal heat conduction from the conductive needle tip, The biopsy needle assembly further comprises a vacuum fluidically coupled to the retractable cannula.
Aspect 9. The biopsy device according to Aspect 8, wherein the handle houses a control unit communicatively coupled to the one or more user input devices, and a signal from the one or more user input devices causes the control unit to operate the conductive needle tip.
Aspect 10. The biopsy device according to any of Aspect 8 to Aspect 9, wherein the retractable cannula comprises a composite material, a ceramic material, or any combination thereof.
Aspect 11. The biopsy device according to any of Aspect 8 to Aspect 10 wherein the stylet comprises a reinforcing rib.
Aspect 12. The biopsy device according to any of Aspect 8 to Aspect 11, further comprises one or more lead wires, wherein the stylet defines one or more wire lumens. The one or more lead wires are positioned within the one or more wire lumens to provide a conductive pathway through the stylet to the conductive needle tip.
Aspect 13. The biopsy device according to any of Aspect 8 to Aspect 12, wherein the conductive needle tip is threaded or crimped into the mounting portion of the stylet.
Aspect 14. The biopsy device according to any of Aspect 8 to Aspect 13, further comprises a heat shield positioned longitudinally between the conductive tip and stylet.
Aspect 15. A method for assembling a biopsy device comprises coupling a conductive needle tip within a mounting portion of a stylet, longitudinally positioning the stylet within a retractable cannula, wherein a sample passage is defined between an inner surface of the retractable cannula and an outer surface of the stylet. The method further comprises retractably attaching the retractable cannula to a handle, wherein the conductive needle tip may be selectively heated, and the stylet substantially insulates proximal heat conduction from the conductive needle tip.
Aspect 16. The biopsy device according to Aspect 15, wherein the conductive needle tip is sandblasted.
Aspect 17. The biopsy device according to any of Aspect 15 to Aspect 16, further comprises coupling the conductive needle tip to a generator power source.
Aspect 18. The biopsy device according to any of Aspect 15 to Aspect 17, wherein the conductive needle tip is configured to heat to a temperature sufficient to cauterize tissue.
Aspect 19. The biopsy device according to any of Aspect 15 to Aspect 18, further comprises coupling the handle comprising one or more user input devices to the conductive needle tip and the retractable cannula.
Aspect 20. The biopsy device according to Aspect 19, wherein the conductive needle tip is operable via actuation of the one or more user input devices.
Aspect 21. A method of performing a biopsy procedure comprises advancing a biopsy device to a targeted biopsy site, The biopsy device comprises a retractable cannula extending distally from a handle and retractable into the handle, a stylet longitudinally positioned within the retractable cannula, a sample passage defined between an inner surface of the retractable cannula and an outer surface of the stylet, the stylet defining a sample recess. The biopsy device further comprises a conductive needle tip coupled to a distal end of the stylet within the mounting portion, wherein operation of the conductive needle tip heats a remaining tissue in contact therewith, and the stylet substantially insulates proximal heat conduction from the conductive needle tip. The method further comprises retracting the retractable cannula, advancing the retractable cannula to obtain a biopsy sample, actuating the conductive needle tip to cauterize the targeted biopsy site.
Aspect 22. The method according to Aspect 21, further comprising coupling the biopsy device to a vacuum source and operating the vacuum source to advance the biopsy sample along the sample passage.
Aspect 23. The method according to Aspect 22, wherein operating the vacuum source advances the biopsy sample to a sample collection basket.
Aspect 24. The method according to Aspect 21, wherein the biopsy device further comprising positioning a heat shield between the conductive tip and stylet.

Although particular embodiments have been disclosed herein in detail, this has been done by way of example for purposes of illustration only, and is not intended to be limiting with respect to the scope of the appended claims, which follow. In particular, it is contemplated that various substitutions, alterations, and modifications may be made without departing from the spirit and scope of the disclosure as defined by the claims. Other aspects, advantages, and modifications are considered to be within the scope of the following claims.

1. A biopsy device comprising:
a handle comprising one or more user input devices; and
a biopsy needle assembly connected to the handle comprising:
a retractable cannula extending distally from the handle and retractable into the handle;
a stylet longitudinally positioned within the retractable cannula, a sample passage defined between an inner surface of the retractable cannula and an outer surface of the stylet, the stylet defining a sample recess; and
a conductive needle tip coupled to a distal end of the stylet within the mounting portion, wherein operation of the conductive needle tip heats a remaining tissue in contact therewith, and the stylet substantially insulates proximal heat conduction from the conductive needle tip.

2. The biopsy device according to claim 1, wherein the handle houses a control unit communicatively coupled to the one or more user input devices, and a signal from the one or more user input devices causes the control unit to operate the conductive needle tip.

3. The biopsy device according to claim 1, wherein the retractable cannula comprises a composite material, a ceramic material, or any combination thereof.

4. The biopsy device according to claim 1, wherein the stylet comprises a reinforcing rib.

5. The biopsy device according to claim 1, further comprising one or more lead wires, wherein:
the stylet defines one or more wire lumens; and
the one or more lead wires are positioned within the one or more wire lumens to provide a conductive pathway through the stylet to the conductive needle tip.

6. The biopsy device according to claim 1, wherein the conductive needle tip is threaded or crimped into the mounting portion of the stylet.

7. The biopsy device according to claim 1, further comprising a heat shield positioned longitudinally between the conductive tip and stylet.

8. A biopsy device comprising:
a handle comprising one or more user input devices; and
a biopsy needle assembly connected to the handle comprising:
a retractable cannula extending distally from the handle and retractable into the handle;
a stylet longitudinally positioned within the retractable cannula, a sample passage defined between an inner surface of the retractable cannula and an outer surface of the stylet, the stylet defining a sample recess;
a conductive needle tip coupled to a distal end of the stylet within the mounting portion, wherein operation of the conductive needle tip heats a remaining tissue in contact therewith, and the stylet substantially insulates proximal heat conduction from the conductive needle tip; and
a vacuum fluidically coupled to the retractable cannula.

9. The biopsy device according to claim 8, wherein the handle houses a control unit communicatively coupled to the one or more user input devices, and a signal from the one or more user input devices causes the control unit to operate the conductive needle tip.

10. The biopsy device according to claim 8, wherein the retractable cannula comprises a composite material, a ceramic material, or any combination thereof.

11. The biopsy device according to claim 8 wherein the stylet comprises a reinforcing rib.

12. The biopsy device according to claim 8, further comprising one or more lead wires, wherein:
the stylet defines one or more wire lumens; and
the one or more lead wires are positioned within the one or more wire lumens to provide a conductive pathway through the stylet to the conductive needle tip.

13. The biopsy device according to claim 8, wherein the conductive needle tip is threaded or crimped into the mounting portion of the stylet.

14. The biopsy device according to claim 8, further comprising a heat shield positioned longitudinally between the conductive tip and stylet.

15. A method for assembling a biopsy device, the method comprising:
coupling a conductive needle tip within a mounting portion of a stylet;
longitudinally positioning the stylet within a retractable cannula, wherein a sample passage is defined between an inner surface of the retractable cannula and an outer surface of the stylet; and
retractably attaching the retractable cannula to a handle, wherein the conductive needle tip may be selectively heated, and the stylet substantially insulates proximal heat conduction from the conductive needle tip.

16. The method according to claim 15, wherein the conductive needle tip is sandblasted.

17. The method according to claim 15, further comprising coupling the conductive needle tip to a generator power source.

18. The method according to claim 15, wherein the conductive needle tip is configured to heat to a temperature sufficient to cauterize tissue.

19. The method of claim 15, further comprising coupling the handle comprising one or more user input devices to the conductive needle tip and the retractable cannula.

20. The method according to claim 19, wherein the conductive needle tip is operable via actuation of the one or more user input devices.

21-24. (canceled)
